(19) United States
(12) Patent Application Publication
(10) Pub. No.: US 2025/0349015 A1
(43) Pub. Date: Nov. 13, 2025

(54) IMAGE REGISTRATION SYSTEM AND METHOD FOR FEMUR SURGERY

(71) Applicants: THE FIRST MEDICAL CENTER OF PLA GENERAL HOSPITAL, Beijing (CN); BEIJING AKSO MEDICAL TECHNOLOGY DEVELOPMENT CO LTD, Beijing (CN)

(72) Inventors: Lihai ZHANG, Beijing (CN); Yang LUO, Beijing (CN); Dezheng SONG, Beijing (CN); Yong LIU, Beijing (CN); Liang LI, Beijing (CN); Nian ZHENG, Beijing (CN); Gongzi ZHANG, Beijing (CN); Shuwei ZHANG, Beijing (CN); Ye PENG, Beijing (CN)

(73) Assignees: THE FIRST MEDICAL CENTER OF PLA GENERAL HOSPITAL, Beijing (CN); BEIJING AKSO MEDICAL TECHNOLOGY DEVELOPMENT CO LTD, Beijing (CN)

(21) Appl. No.: 18/547,301

(22) PCT Filed: Jul. 21, 2022
(86) PCT No.: PCT/CN2022/106960

§ 371 Date: Oct. 22, 2024

(30) Foreign Application Priority Data
Jul. 21, 2022 (WO) PCT/CN2022/106960

(51) Int. Cl. G06T 7/33; A61B 34/10; G06T 7/00; G06T 7/13; G06T 11/00

(52) U.S. Cl. CPC G06T 7/344; A61B 34/10; G06T 7/0012; G06T 7/13; G06T 11/008; A61B 2034/105; G06T 2207/10081; G06T 2207/10116; G06T 2207/30008; G06T 2210/41

(57) ABSTRACT

An image registration system and a method for a femur surgery relates to the field of surgery image registration are provided, before a surgery, DRR projections at different angles are carried out of a three-dimensional femur CT image, femoral coordinate systems and femoral characteristic points are respectively generated and selected on a the two-dimensional DRR projection images and two-dimensional X-ray images. Based on the femoral coordinate systems and the femoral characteristic points, the two-dimensional DRR projection images and the two-dimensional X-ray images are registered, so that the image registration precision is improved, a two-dimensional DRR projection image in the same femoral posture as a femoral posture during a two-dimensional X-ray image scanning can be accurately obtained, which can obtain a real surgical path for surgical navigation, ensures the precision of surgery-assisted navigation, and further improves the surgery quality.

### CROSS-REFERENCE TO RELATED APPLICATION

This patent application is a National Stage of International Application No. PCT/CN2022/106960, filed Jul. 21, 2022 which is hereby incorporated by reference in their entireties.

### TECHNICAL FIELD

The present disclosure relates to the field of surgery image registration, in particular to an image registration system and a method for a femur surgery.

### BACKGROUND

The femoral neck needs to undertake the movement and support functions of the human body during most activities, and the treatment effect of the femoral neck fracture will directly affect the postoperative life quality of the patient. At present, the femoral neck fracture accounts for 3.6 percent of the total fracture, accounts for half of the hip fracture, is frequently generated in middle-aged and elderly people with osteoporosis, the incidence rate of the fracture is gradually increased year by year along with the serious aging of the society, and the surgery navigation system has important significance in assisting the surgery of the femoral neck fracture.

At present, the navigation modes commonly used in femur surgery according to different medical image data in the market are mainly divided into the following four modes: 1) a preoperative navigation system based on CT data; 2) an intraoperative imaging navigation system based on a C-shaped arm X-ray machine; 3) a navigation system based on intraoperative three-dimensional reconstruction; and 4) a navigation system based on three-dimensional-two-dimensional image registration.

The preoperative navigation system based on CT data cannot perform intraoperative spatial positioning due to no intraoperative information support, the intraoperative imaging navigation system based on the C-shaped arm X-ray machine is not visual enough, and the navigation system based on intraoperative three-dimensional reconstruction is high in price and low in popularization rate. The navigation system based on three-dimensional-two-dimensional image registration can effectively avoid the above defects, and its difficulty lies in accurate registration of the three-dimensional image and the two-dimensional image.

### SUMMARY

The present disclosure aims to provide an image registration system and method for a femur surgery, which improves the precision of image registration, ensures the precision of surgery-assisted navigation and further improves the surgery quality.

In order to achieve the above purposes, the present disclosure provides the following scheme.

An image registration system for a femur surgery, including: a projection module, a preprocessing module and a registration module;
the projection module is configured to carry out DRR projections at different angles of a three-dimensional femur CT image, to obtain a plurality of two-dimensional DRR projection images;
the preprocessing module is configured to preprocess femur images in the two-dimensional DRR projection images and two-dimensional X-ray images, to generate femoral coordinate systems and femoral characteristic points; and the two-dimensional X-ray images include a plurality of X-ray plane images taken from different angles of a femoral; and
the registration module is configured to determine, from the plurality of two-dimensional DRR projection images, a two-dimensional DRR projection image in a same femoral posture as a femoral posture of a two-dimensional X-ray image according to the femoral coordinate systems and the femoral characteristic points of the two-dimensional DRR projection images and the two-dimensional X-ray images, to obtain a plurality of target DRR projection images.

Optionally, the femur includes a femoral head, a femoral neck and a femoral shaft, and the preprocessing module includes a femoral contour line generating unit, a femoral head fitting unit, a femoral neck characteristic line selecting unit, a femoral coordinate system establishing unit, a femoral characteristic circle generating unit, a femoral characteristic line selecting unit and a femoral characteristic point selecting unit;
the femoral contour line generating unit is configured to automatically extract a femoral outer contour, to obtain a femoral contour line;
the femoral head fitting unit is configured to fit the femoral head into a circle, to obtain a femoral head fitting circle;
the femoral neck characteristic line selecting unit is configured to select a shortest distance connection line constituted by two points located at fitting curves of two femoral neck contour lines at the femoral neck as a femoral neck characteristic line;
the femoral coordinate system establishing unit is configured to establish a femoral coordinate system by using a connection line from a center of the femoral head fitting circle to a midpoint of the femoral neck characteristic line as a coordinate axis of the femoral coordinate system;
the femoral characteristic circle generating unit is configured to respectively generate a first characteristic circle and a second characteristic circle by using a radius of the femoral head fitting circle as a radius and selecting two points located on an extension line extended from a connection line from the center of the femoral head fitting circle to the midpoint of the femoral neck characteristic line as centers of circles; and the first characteristic circle is tangent to the femoral head fitting circle, and a center of the first characteristic circle is on an edge line of the second characteristic circle;
the femoral characteristic line selecting unit is configured to select a plurality of femoral characteristic lines perpendicular to the connection line from the center of the femoral head fitting circle to the midpoint of the femoral neck characteristic line; and
the femoral characteristic point selecting unit is configured to select intersection points of all femoral characteristic lines and the femoral contour lines as femoral characteristic points.

Optionally, the femoral characteristic line selecting unit includes: a first femoral characteristic line selecting subunit, a second femoral characteristic line selecting subunit, a third femoral characteristic line selecting subunit, a fourth femoral characteristic line selecting subunit and a fifth femoral characteristic line selecting subunit;
the first femoral characteristic line selecting subunit is configured to select a first femoral characteristic line; and the first femoral characteristic line is a line segment that passes through a tangent point of the femoral head fitting circle and the first characteristic circle;
the second femoral characteristic line selecting subunit is configured to select a second femoral characteristic line; and the second femoral characteristic line is a line segment that passes through the center of the first characteristic circle;
the third femoral characteristic line selecting subunit is configured to select a third femoral characteristic line; and the third femoral characteristic line is a line segment that passes through a center of the second characteristic circle;
the fourth femoral characteristic line selecting subunit is configured to select a fourth femoral characteristic line; and the fourth femoral characteristic line is parallel to the second femoral characteristic line and is a line segment of the tangent line of the second characteristic circle; and
the fifth femoral characteristic line selecting subunit is configured to select a fifth femoral characteristic line; and the fifth femoral characteristic line is a line segment which passes through a midpoint of a connection line from an intersection point of an extension line and the third femoral characteristic line to an intersection point of an extension line and the fourth femoral characteristic line, where the extension line is extended from the connection line from the center of the femoral head fitting circle to the midpoint of the femoral neck characteristic line.

Optionally, the registration module includes a femoral coordinate system overlap unit and a femoral characteristic point registration unit;
the femoral coordinate system overlap unit is configured to overlap the femoral coordinate systems of the two-dimensional DRR projection images and the two-dimensional X-ray images; and
the femoral characteristic point registration unit is configured to match positions of the femoral characteristic points of the two-dimensional DRR projection images and the two-dimensional X-ray images in a one-to-one correspondence.

Optionally, the preprocessing module further includes: an interested region selecting unit;
the interested region selecting unit is configured to select interested regions composed of line segments and arcs at positions of a femoral head contour line and two femoral neck contour lines of the femoral contour lines respectively.

On the other hand, in view of the above image registration system, the present disclosure also provide an image registration method for a femoral surgery, including the following steps of:
carrying out DRR projection at different angles of a three-dimensional femur CT image, to obtain a plurality of two-dimensional DRR projection images;
preprocessing femur images in the two-dimensional DRR projection images and two-dimensional X-ray images, to generate femoral coordinate systems and femoral characteristic points; where the two-dimensional X-ray image include a plurality of X-ray plane images taken from different angles of a femoral; and
determining, from the plurality of two-dimensional DRR projection images, a two-dimensional DRR projection image in the same femoral posture as a femoral posture of a two-dimensional X-ray image according to the femoral coordinate systems and the femoral characteristic points of the two-dimensional DRR projection images and the two-dimensional X-ray images, to obtain a plurality of target DRR projection images.

Optionally, the femur includes a femoral head, a femoral neck and a femoral shaft, and the preprocessing femur images in the two-dimensional DRR projection images and two-dimensional X-ray images, to generate femoral coordinate systems and femoral characteristic points specifically includes:
automatically extracting a femoral outer contour, to obtain a femoral contour line;
fitting the femoral head into a circle, to obtain a femoral head fitting circle;
selecting a shortest distance connection line constituted by two points located at fitting curves of two femoral neck contour lines at the femoral neck as a femoral neck characteristic line;
establish a femoral coordinate system by using a connection line from a center of the femoral head fitting circle to a midpoint of the femoral neck characteristic line as a coordinate axis of the femoral coordinate system;
generating a first characteristic circle and a second characteristic circle respectively by using a radius of the femoral head fitting circle as a radius and selecting two points located on an extension line extended from a connection line from the center of the femoral head fitting circle to the midpoint of the femoral neck characteristic line as centers of circles, where the first characteristic circle is tangent to the femoral head fitting circle, and a center of the first characteristic circle is on an edge line of the second characteristic circle;
generating a plurality of femoral characteristic lines perpendicular to the connection line from the center of the femoral head fitting circle to the midpoint of the femoral neck characteristic line; and
selecting intersection points of all femoral characteristic lines and the femoral contour lines as femoral characteristic points.

Optionally, the generating a plurality of femoral characteristic lines perpendicular to the connection line from the center of the femoral head fitting circle to the midpoint of the femoral neck characteristic line specifically includes:
selecting a line segment that passes through a tangent point of the femoral head fitting circle and the first characteristic circle as a first femoral characteristic line;
selecting a line segment that passes through the center of the first characteristic circle as a second femoral characteristic line;
selecting a line segment that passes through a center of the second characteristic circle as a third femoral characteristic line;
selecting a line segment, which is parallel to the second femoral characteristic line and is the tangent line of the second characteristic circle, as a fourth femoral characteristic line; and

selecting a line segment, which passes through a midpoint of a connection line from an intersection point of an extension line and the third femoral characteristic line to an intersection point of an extension line and the fourth femoral characteristic line, as a fifth femoral characteristic line, where the extension line is extended from the connection line from the center of the femoral head fitting circle to the midpoint of the femoral neck characteristic line.

Optionally, the determining, from the plurality of two-dimensional DRR projection images, a two-dimensional DRR projection image in the same femoral posture as a femoral posture of a two-dimensional X-ray image according to the femoral coordinate systems and the femoral characteristic points of the two-dimensional DRR projection images and the two-dimensional X-ray images, to obtain a plurality of target DRR projection images specifically includes:
overlapping the femoral coordinate systems of the two-dimensional DRR projection images and the two-dimensional X-ray images; and
matching positions of the femoral characteristic points of the two-dimensional DRR projection images and the two-dimensional X-ray images in a one-to-one correspondence.

Optionally, the method further includes:
selecting interested regions composed of line segments and arcs at positions of a femoral head contour line and two femoral neck contour lines of the femoral contour lines respectively; and
identifying contour lines in the interested regions, to generate fitting curves of the femoral head contour line and two femoral neck contour lines;
automatically generating a femoral head fitting circle according to the fitting curves of the femoral contour line at the femoral head; and
selecting a shortest distance connection line constituted by two points located at fitting curves of two femoral neck contour lines at the femoral neck as a femoral neck characteristic line.

According to the specific embodiment provided by the present disclosure, the present disclosure discloses the following technical effects: the present disclosure provides an image registration system and method for a femur surgery, including: a projection module, a preprocessing module and a registration module; the projection module is configured to carry out DRR projections at different angles on a three-dimensional femur CT image, to obtain a plurality of two-dimensional DRR projection images; the projection module is configured to carry out DRR projections at different angles of a three-dimensional femur CT image, to obtain a plurality of two-dimensional DRR projection images; the two-dimensional X-ray images include a plurality of X-ray plane images taken from different angles of a femur; and the registration module is configured to determine, from the plurality of two-dimensional DRR projection images, a two-dimensional DRR projection image in the same femoral posture as a femoral posture of a two-dimensional X-ray image according to the femoral coordinate systems and the femoral characteristic points of the two-dimensional DRR projection images and the two-dimensional X-ray images, to obtain a plurality of target DRR projection images. In the present disclosure, before a surgery, DRR projections at different angles are carried out on a three-dimensional femoral CT image, femoral coordinate systems and femoral characteristic points are respectively generated and selected on a the two-dimensional DRR projection images and two-dimensional X-ray images. Based on the femoral coordinate systems and the femoral characteristic points, the two-dimensional DRR projection images and the two-dimensional X-ray images are registered, so that the image registration precision is improved, a two-dimensional DRR projection image in the same femoral posture as a femoral posture of a two-dimensional X-ray image can be accurately obtained, which can obtain a real surgical path for surgical navigation, ensures the precision of surgery-assisted navigation, and further improves the surgery quality.

### BRIEF DESCRIPTION OF THE DRAWINGS

In order to more clearly illustrate the embodiments of the present disclosure or the technical solutions in the prior art, the drawings needed in the embodiments will be briefly described below, and it is obvious that the drawings in the following description are only some embodiments of the present disclosure, and it is obvious for those skilled in the art to obtain other drawings without creative efforts.

FIG. 1 is a block diagram of image registration system for a femur surgery according to the present disclosure.

FIG. 2 is a block diagram of a femoral characteristic line selecting unit in the image registration system according to the present disclosure.

FIG. 3 is a flowchart of an image registration method for a femur surgery according to the present disclosure.

FIG. 4 is a detailed flowchart of step S2 in the image registration method according to the present disclosure.

FIG. 5 is a detailed flowchart of step S26 in the image registration method according to the present disclosure.

FIG. 6 is three-dimensional femur CT images reconstructed in the image registration method according to the present disclosure.

FIG. 7 is schematic diagrams of femoral contour lines generated by two-dimensional DRR projection images in the image registration method according to the present disclosure.

FIG. 8 is schematic diagrams of femoral characteristic points generated for two-dimensional DRR projection diagrams in the image registration method according to the present disclosure.

FIG. 9 is two-dimensional X-ray images scanned in an image registration method according to the present disclosure.

FIG. 10 is schematic diagrams of femoral contour lines generated for two-dimensional X-ray images in the image registration method according to the present disclosure.

FIG. 11 is a schematic diagram of femoral feature points generated on two-dimensional X-ray images in the image registration method according to the present disclosure.

In the drawings: 1, projection module; 2, preprocessing module; 3, registration module; 21, femoral contour line generating unit; 22, femoral head fitting unit; 23, femoral neck characteristic line selecting unit; 24, femoral coordinate system establishing unit; 25, femoral characteristic circle generating unit; 26, femoral characteristic line selecting unit; 27, femoral characteristic point selecting unit; 28, interested region selecting unit; 261, first femoral characteristic line selecting subunit; 262, second femoral characteristic line selecting subunit; 263, third femoral characteristic line selecting subunit; 264, fourth femoral characteristic line selecting subunit; 265, fifth femoral characteristic line selecting subunit; 31, femoral coordinate system overlap unit; 32, femoral characteristic point registration unit.

### DETAILED DESCRIPTION OF THE EMBODIMENTS

The technical solutions in the embodiments of the present disclosure will be clearly and completely described below with reference to the drawings in the embodiments of the present disclosure. Obviously, the described embodiments are only a part of the embodiments of the present disclosure, and not all of the embodiments. All other embodiments, which can be derived by a person skilled in the art from the embodiments given herein without making any creative effort, shall fall within the protection scope of the present disclosure.

The present disclosure aims to provide an image registration system and method for a femur surgery, which improves the precision of image registration, ensures the precision of surgery-assisted navigation and further improves the surgery quality.

In order to make the aforementioned objects, features and advantages of the present disclosure comprehensible, embodiments accompanied with figures are described in further detail below.

### Embodiment 1

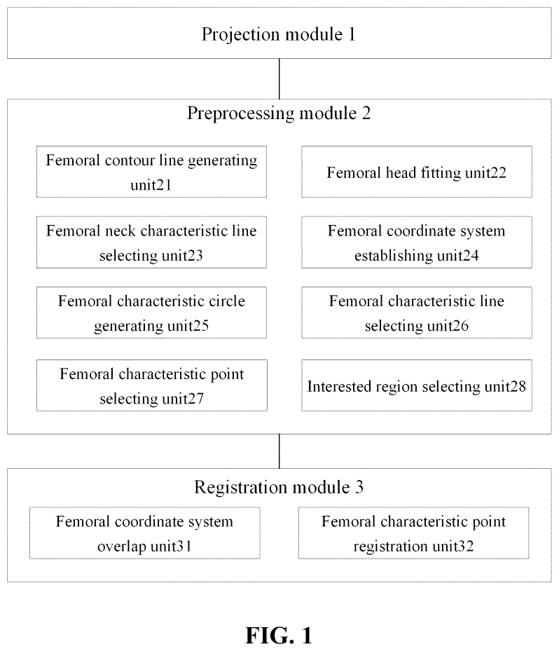

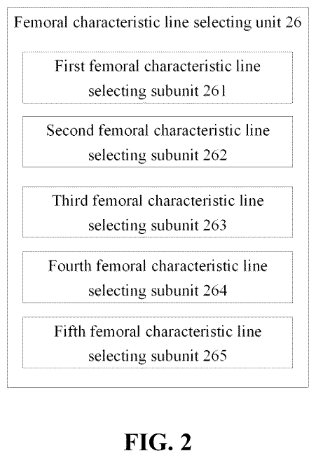

The present disclosure provides an image registration system for a femur surgery, as shown in FIGS. 1-2, the image registration system for a femur surgery of the present disclosure includes: a projection module 1, a preprocessing module 2 and a registration module 3.

The projection module 1 is configured to carry out DRR projections at different angles of a three-dimensional femur CT image, to obtain a plurality of two-dimensional DRR projection images.

The preprocessing module 2 is configured to preprocess femur images in the two-dimensional DRR projection images and two-dimensional X-ray images, to generate femoral coordinate systems and femoral characteristic points; the two-dimensional X-ray images are a plurality of X-ray plane images taken from different angles of a femur.

The registration module 3 is configured to determine, from the plurality of two-dimensional DRR projection images, a two-dimensional DRR projection image in a same femoral posture as a femoral posture of a two-dimensional X-ray image according to the femoral coordinate systems and the femoral characteristic points of the two-dimensional DRR projection images and the two-dimensional X-ray images, to obtain a plurality of target DRR projection images.

The femur includes a femoral head, a femoral neck and a femoral shaft, in this embodiment, the preprocessing module 2 includes a femoral contour line generating unit 21, a femoral head fitting unit 22, a femoral neck characteristic line selecting unit 23, a femoral coordinate system establishing unit 24, a femoral characteristic circle generating unit 25, a femoral characteristic line selecting unit 26 and a femoral characteristic point selecting unit 27.

The femoral contour line generating unit 21 is configured to automatically extract a femoral outer contour, to obtain a femoral contour line.

The femoral head fitting unit 22 is configured to fit the femoral head into a circle, to obtain a femoral head fitting circle.

The femoral neck characteristic line selecting unit 23 is configured to select a shortest distance connection line constituted by two points located at fitting curves of two femoral neck contour lines at the femoral neck as a femoral neck characteristic line.

The femoral coordinate system establishing unit 24 is configured to establish a femoral coordinate system by using a connection line from a center of the femoral head fitting circle to a midpoint of the femoral neck characteristic line as a coordinate axis of the femoral coordinate system.

The femoral characteristic circle generating unit 25 is configured to respectively generate a first characteristic circle and a second characteristic circle by using a radius of the femoral head fitting circle as a radius and selecting two points located on an extension line extended from a connection line from the center of the femoral head fitting circle to the midpoint of the femoral neck characteristic line as centers of circles; and the first characteristic circle is tangent to the femoral head fitting circle, and a center of the first characteristic circle is on an edge line of the second characteristic circle;

The femoral characteristic line selecting unit 26 is configured to select a plurality of femoral characteristic lines perpendicular to the connection line from the center of the femoral head fitting circle to the midpoint of the femoral neck characteristic line.

The femoral characteristic point selecting unit 27 is configured to select intersection points of all femoral characteristic lines and the femoral contour lines as femoral characteristic points.

In this embodiment, the femoral characteristic line selecting unit 26 includes: a first femoral characteristic line selecting subunit 261, a second femoral characteristic line selecting subunit 262, a third femoral characteristic line selecting subunit 263, a fourth femoral characteristic line selecting subunit 264 and a fifth femoral characteristic line selecting subunit 265.

The first femoral characteristic line selecting subunit 261 is configured to selecting a first femoral characteristic line; and the first femoral characteristic line is a line segment that passes through a tangent point of the femoral head fitting circle and the first characteristic circle.

The second femoral characteristic line selecting subunit 262 is configured to select a second femoral characteristic line; and the second femoral characteristic line is a line segment that passes through the center of the first characteristic circle.

The third femoral characteristic line selecting subunit 263 is configured to select a third femoral characteristic line; and the third femoral characteristic line is a line segment that passes through a center of the second characteristic circle.

The fourth femoral characteristic line selecting subunit 264 is configured to select a fourth femoral characteristic line; and the fourth femoral characteristic line is parallel to the second femoral characteristic line and is a line segment of the tangent line of the second characteristic circle.

The fifth femoral characteristic line selecting subunit 265 is configured to select a fifth femoral characteristic line; and the fifth femoral characteristic line is a line segment which passes through a midpoint of a connection line from an intersection point of an extension line and the third femoral characteristic line to an intersection point of an extension line and the fourth femoral characteristic line, where the extension line is extended from the connection line from the center of the femoral head fitting circle to the midpoint of the femoral neck characteristic line.

Specifically, in this embodiment, the registration module 3 includes: a femoral coordinate system overlap unit 31 and a femoral characteristic point registration unit 32.

The femoral coordinate system overlap unit 31 is configured to overlap the femoral coordinate systems of the two-dimensional DRR projection images and the two-dimensional X-ray images.

The femoral characteristic point registration unit 32 is configured to match positions of the femoral characteristic points of the two-dimensional DRR projection images and the two-dimensional X-ray images in a one-to-one correspondence.

In order to reduce the amount of computation, in this embodiment, the preprocessing module 2 further includes: an interested region selecting unit 28.

The interested region selecting unit 28 is configured to respectively select interested regions composed of line segments and arcs at positions of a femoral head contour line and two femoral neck contour lines of the femoral contour lines. The selection of interested region is carried out on the femoral head and the femoral neck by the selection template consisting of the line segment and the circular arc, which eliminates the need for a contour recognition of the whole region of X-ray image, thus reducing the amount of calculation.

### Embodiment 2

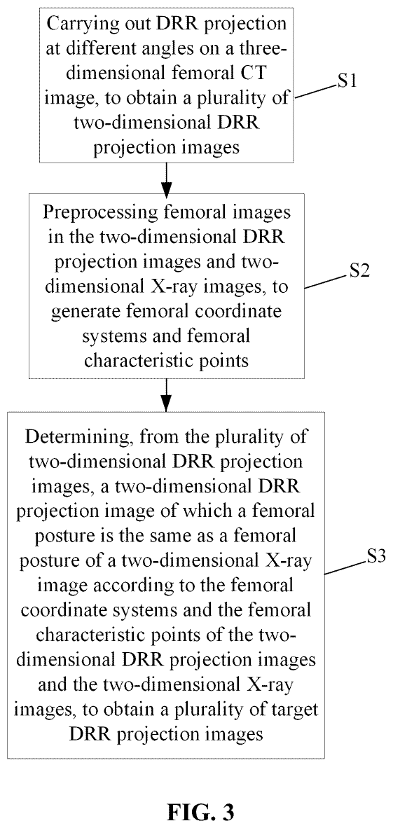

The present disclosure also provides an image registration method for a femur surgery, corresponding to the image registration system for a femur surgery in Embodiment 1. As shown in FIG. 3, the image registration method including the steps of:
S1, carrying out DRR projection at different angles on a three-dimensional femur CT image, to obtain a plurality of two-dimensional DRR projection images;
S2, preprocessing femur images in the two-dimensional DRR projection images and two-dimensional X-ray images, to generate femoral coordinate systems and femoral characteristic points; where the two-dimensional X-ray images include a plurality of X-ray plane images taken from different angles of a femur; and
S3, determining, from the plurality of two-dimensional DRR projection images, a two-dimensional DRR projection image in a same femoral posture as a femoral posture of a two-dimensional X-ray image according to the femoral coordinate systems and the femoral characteristic points of the two-dimensional DRR projection images and the two-dimensional X-ray images, to obtain a plurality of target DRR projection images. Step S3 specifically includes:
S31, overlapping the femoral coordinate systems of the two-dimensional DRR projection images and the two-dimensional X-ray images;
S32, matching positions of the femoral characteristic points of the two-dimensional DRR projection images and the two-dimensional X-ray images one-by-one.

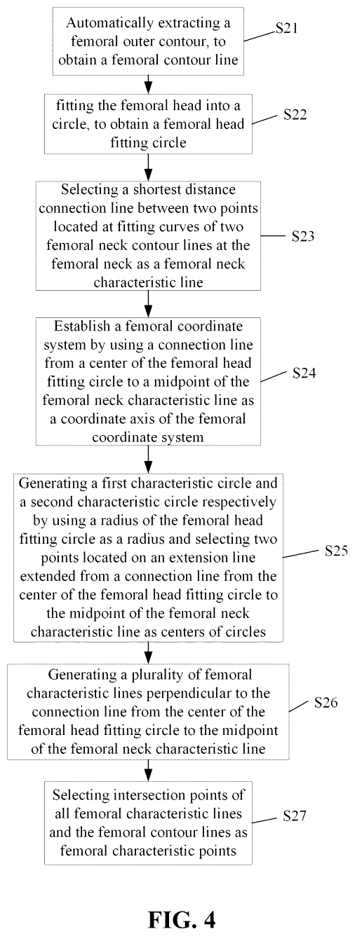

The femur includes a femoral head, a femoral neck and a femoral shaft, and only one part of the femoral shaft can be displayed by the CT or X-ray image. In this embodiment, as shown in FIG. 4, Step S2 specifically includes:
S21, automatically extracting a femoral outer contour, to obtain a femoral contour line;
S22, fitting the femoral head into a circle, to obtain a femoral head fitting circle;
S23, selecting a shortest distance connection line constituted by two points located at fitting curves of two femoral neck contour lines at the femoral neck as a femoral neck characteristic line;
S24, establishing a femoral coordinate system by using a connection line from a center of the femoral head fitting circle to a midpoint of the femoral neck characteristic line as a coordinate axis of the femoral coordinate system;
S25, generating a first characteristic circle and a second characteristic circle respectively by using a radius of the femoral head fitting circle as a radius and selecting two points located on an extension line extended from a connection line from the center of the femoral head fitting circle to the midpoint of the femoral neck characteristic line as centers of circles, where the first characteristic circle is tangent to the femoral head fitting circle, and a center of the first characteristic circle is on an edge line of the second characteristic circle;
S26, generating a plurality of femoral characteristic lines perpendicular to the connection line from the center of the femoral head fitting circle to the midpoint of the femoral neck characteristic line; and
S27, selecting intersection points of all femoral characteristic lines and the femoral contour lines as femoral characteristic points.

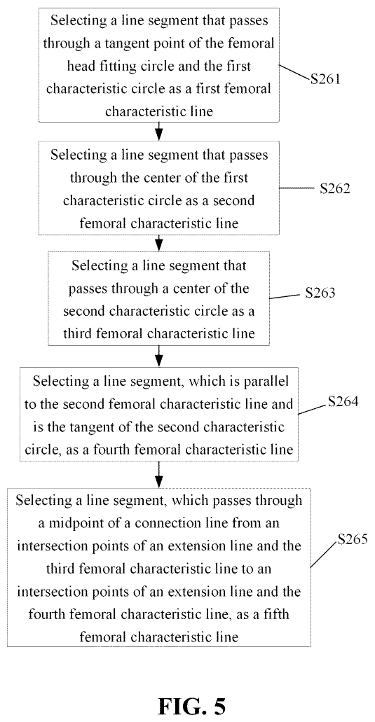

Regarding the selection rule of each femoral characteristic line, in this embodiment, as shown in FIG. 5, Step S26 specifically includes:
S261, selecting a line segment that passes through a tangent point of the femoral head fitting circle and the first characteristic circle as a first femoral characteristic line;
S262, selecting a line segment that passes through the center of the first characteristic circle as a second femoral characteristic line;
S263, selecting a line segment that passes through a center of the second characteristic circle as a third femoral characteristic line;
S264, selecting a line segment, which is parallel to the second femoral characteristic line and is the tangent line of the second characteristic circle, as a fourth femoral characteristic line; and
S265, selecting a line segment, which passes through a midpoint of a connection line from an intersection point of an extension line and the third femoral characteristic line to an intersection point of an extension line and the fourth femoral characteristic line, as a fifth femoral characteristic line, where the extension line is extended from the connection line from the center of the femoral head fitting circle to the midpoint of the femoral neck characteristic line.

In order to reduce the amount of calculation, the image registration method of the present embodiment further includes:
A1, respectively selecting interested regions composed of line segments and arcs at positions of a femoral head contour line and two femoral neck contour lines of the femoral contour lines. The selection of interested regions is carried out on the femoral head and the femoral neck by the selection template consisting of the line segment and the circular arc, which eliminates the need for a contour recognition of the whole region of X-ray image, thus reducing the amount of calculation;
A2, identifying contour lines in the interested regions, to generate fitting curves of the femoral head contour line and two femoral neck contour lines;
A3, automatically generating a femoral head fitting circle according to the fitting curves of the femoral contour line at the femoral head; and
A4, selecting a shortest distance connection line constituted by two points located at fitting curves of two femoral neck contour lines at the femoral neck as a femoral neck characteristic line.

The following describes the image registration method for a femur surgery in further detail with reference to a specific example, including the following steps.

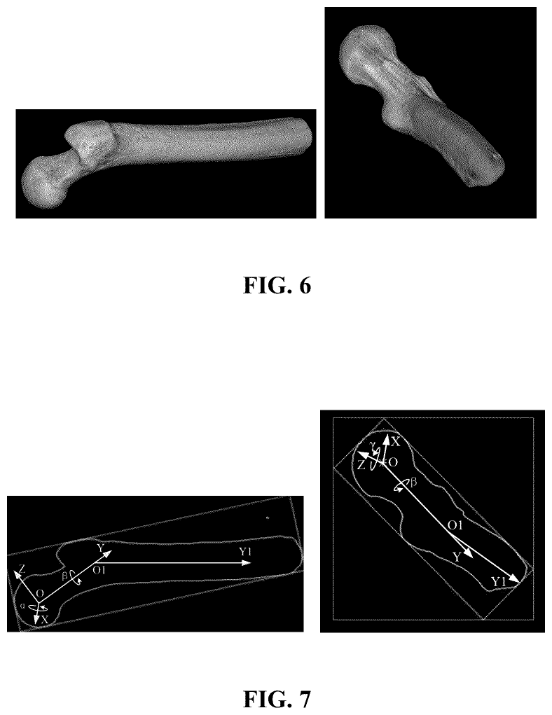

1) Before a surgery is started, a CT imaging device obtains an image data of a femur of a patient and generates a three-dimensional femur CT image through processing and reconstruction, as shown in FIG. 6. A surgery path is planned on the three-dimensional femur CT image.

2) According to the surgery requirement, the three-dimensional femur CT image is projected for a plurality of times in the lateral single angle Z direction, as shown in the left side of FIG. 7. O is the center of a femoral head fitting circle, OY passes through a femoral neck characteristic line and is intersected with a central axis of a femoral shaft at O1, OZ is perpendicular to OY and is in an OO1Y1 plane, and an OX axis conforms to the right-hand rule.

3) A preset degree is rotated around the X and Y axes at intervals, α and β are within the range of 1-2 degrees, a plurality of DRR images projected from Z-axis are generated, to obtain corresponding pose parameters P=(θₓ, θ_{y}, θ_{z}, Pₓ, P_{y}, P_{z}); the θₓ, θ_{y} and θ_{z} respectively represent the rotation angle in a reference coordinate system; the Pₓ, P_{y} and P_{z} respectively represent the translation amount along the three main axes under the reference coordinate system; similarly, a plurality of projections are also taken in the X direction of the normal single image angle, as shown in the right side of FIG. 7, multiple two-dimensional DRR projection images are obtained.

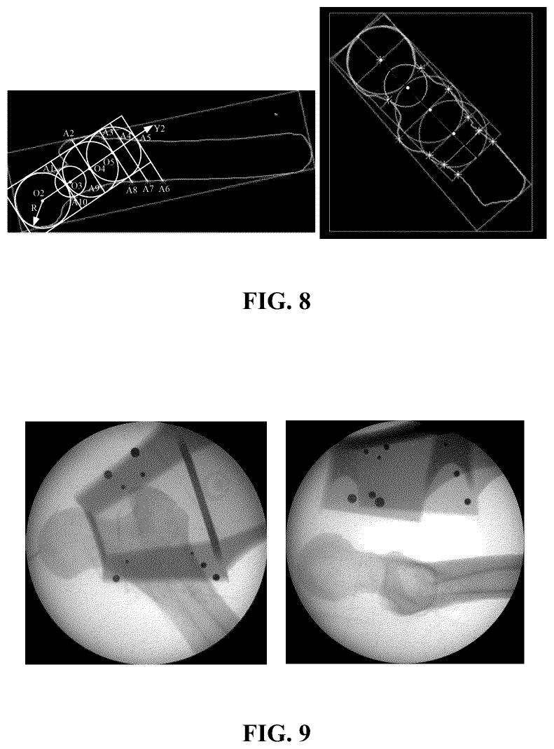

4) The two-dimensional DRR projection images are processed, as shown in FIG. 8. femoral contour lines are automatically extracted, and femoral head fitting circles are automatically generated, where the centers O2, O3 of the circles are the midpoints of the femoral neck characteristic line, a first characteristic circle O4 and a second characteristic circle O5 are drawn on the extension line of O2O3, and the femoral characteristic points A1, A2, A3, A4, A5, A6, A7, A8, A9 and A10 generated by intersecting five femoral characteristic lines with the femoral contour line are selected.

5) When the patient lies down on an operating table to perform the surgery, two-dimensional X-ray images shot by the C-shaped arm X-ray machine at different angles are obtained, as shown in FIG. 9, the included angle between shooting angles of two two-dimensional images is 90 degrees, the left side of FIG. 9 shows a normal X-ray image, namely, X-rays are shot vertically while the patient lies down, and the right side of FIG. 9 shows a side X-ray image, namely, X-rays are shot horizontally while the patient lies down.

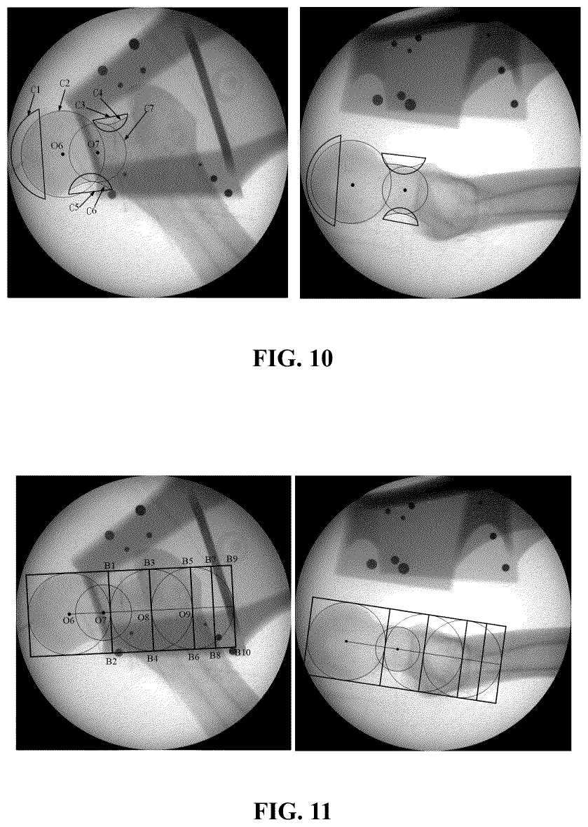

6) As shown in FIG. 10, taking the normal X-ray image as an example, an interested region C1 is defined at the femoral head to automatically generate a femoral head fitting circle C2, or three points are manually selected in the area C1 to generate a femoral head fitting circle C2 with a center O6. Interested regions C3 and C5 of the femoral neck are manually selected, and femoral neck outer contour fitting curves C4 and C6 automatically generated. Two fitting curves at the narrowest part of the femoral neck are manually selected, the minimum distance of the two fitting curves is calculated, or two points at the narrowest part of the femoral neck by visual inspection are manually selected, a minimum distance is taken as a femoral neck characteristic line, and the femoral neck characteristic line is taken as the diameter to generate a femoral neck minimum circle C7 with a center O7. Referring to Step 4), the femoral coordinate systems, the femoral feature circles, the femoral feature lines and the femoral characteristic points are automatically generated for the two-dimensional X-ray image, as shown in FIG. 11.

7) Each of anterior and lateral two-dimensional DRR projection images of the femur are automatically registered with anterior and lateral two-dimensional X-ray images of the femur, the two-dimensional DRR projections are overlapped with the femoral coordinate systems of the two-dimensional X-ray images, all of the distances between corresponding femoral characteristic points are calculated, and the similarity of the characteristic points are compared. If a registration result of the two-dimensional DRR projections and the two-dimensional X-ray images is within an error range, a projection pose parameter of the two-dimensional DRR projection image is obtained. If the registration results are more than one group, a group with a smallest error is selected to complete the registration of the three-dimensional femur CT image and the two-dimensional X-ray images.

8) The surgery path having been accurately registered are outputted by using a biplane method according to the projection pose parameter of the two-dimensional DRR projection image and the surgery planning path information.

The principles and embodiments of the present disclosure have been described herein using specific examples, which are provided only to help understand the method and the core concept of the present disclosure. Meanwhile, for a person skilled in the art, according to the idea of the present disclosure, the specific embodiments and the application range may be changed. In view of the above, the present disclosure should not be construed as limiting the present disclosure.

What is claimed is:

1. An image registration system for a femur surgery, comprising: a projection module, a preprocessing module and a registration module;
the projection module is configured to carry out DRR projections at different angles of a three-dimensional femur CT image, to obtain a plurality of two-dimensional DRR projection images;
the preprocessing module is configured to preprocess femur images in the two-dimensional DRR projection images and two-dimensional X-ray images, to generate femoral coordinate systems and femoral characteristic points; and the two-dimensional X-ray images include a plurality of X-ray plane images taken from different angles of a femur; and
the registration module is configured to determine, from the plurality of two-dimensional DRR projection images, a two-dimensional DRR projection image in a same femoral posture as a femoral posture of the two-dimensional X-ray images according to the femoral coordinate systems and the femoral characteristic points of the two-dimensional DRR projection images and the two-dimensional X-ray images, to obtain a plurality of target DRR projection images.

2. The image registration system according to claim 1, wherein the femur comprises a femoral head, a femoral neck and a femoral shaft, and the preprocessing module comprises a femoral contour line generating unit, a femoral head fitting unit, a femoral neck characteristic line selecting unit, a femoral coordinate system establishing unit, a femoral characteristic circle generating unit, a femoral characteristic line selecting unit and a femoral characteristic point selecting unit;
the femoral contour line generating unit is configured to automatically extract a femoral outer contour, to obtain a femoral contour line;
the femoral head fitting unit is configured to fit the femoral head into a circle, to obtain a femoral head fitting circle;
the femoral neck characteristic line selecting unit is configured to select a shortest distance connection line constituted by two points located at fitting curves of two femoral neck contour lines at the femoral neck as a femoral neck characteristic line;
the femoral coordinate system establishing unit is configured to establish a femoral coordinate system by using a connection line from a center of the femoral head fitting circle to a midpoint of the femoral neck characteristic line as a coordinate axis of the femoral coordinate system;
the femoral characteristic circle generating unit is configured to respectively generate a first characteristic circle and a second characteristic circle by using a radius of the femoral head fitting circle as a radius and selecting two points located on an extension line extended from a connection line from the center of the femoral head fitting circle to the midpoint of the femoral neck characteristic line as centers of circles; and the first characteristic circle is tangent to the femoral head fitting circle, and a center of the first characteristic circle is on an edge line of the second characteristic circle;
the femoral characteristic line selecting unit is configured to select a plurality of femoral characteristic lines perpendicular to the connection line from the center of the femoral head fitting circle to the midpoint of the femoral neck characteristic line; and
the femoral characteristic point selecting unit is configured to select intersection points of all femoral characteristic lines and the femoral contour lines as femoral characteristic points.

3. The image registration system according to claim 2, wherein the femoral characteristic line selecting unit comprises: a first femoral characteristic line selecting subunit, a second femoral characteristic line selecting subunit, a third femoral characteristic line selecting subunit, a fourth femoral characteristic line selecting subunit and a fifth femoral characteristic line selecting subunit;
the first femoral characteristic line selecting subunit is configured to select a first femoral characteristic line; and the first femoral characteristic line is a line segment that passes through a tangent point of the femoral head fitting circle and the first characteristic circle;
the second femoral characteristic line selecting subunit is configured to select a second femoral characteristic line; and the second femoral characteristic line is a line segment that passes through the center of the first characteristic circle;
the third femoral characteristic line selecting subunit is configured to select a third femoral characteristic line; and the third femoral characteristic line is a line segment that passes through a center of the second characteristic circle;
the fourth femoral characteristic line selecting subunit is configured to select a fourth femoral characteristic line; and the fourth femoral characteristic line is parallel to the second femoral characteristic line and is a line segment of the tangent line of the second characteristic circle; and
the fifth femoral characteristic line selecting subunit is configured to select a fifth femoral characteristic line; and the fifth femoral characteristic line is a line segment which passes through a midpoint of a connection line from an intersection point of an extension line and the third femoral characteristic line to an intersection point of the extension line and the fourth femoral characteristic line, wherein the extension line is extended from the connection line from the center of the femoral head fitting circle to the midpoint of the femoral neck characteristic line.

4. The image registration system according to claim 3, wherein the registration module comprises a femoral coordinate system overlap unit and a femoral characteristic point registration unit;
the femoral coordinate system overlap unit is configured to overlap the femoral coordinate systems of the two-dimensional DRR projection images and the two-dimensional X-ray images; and
the femoral characteristic point registration unit is configured to match positions of the femoral characteristic points of the two-dimensional DRR projection images and the two-dimensional X-ray images in a one-to-one correspondence.

5. The image registration system according to claim 2, wherein the preprocessing module further comprises: an interested region selecting unit;
the interested region selecting unit is configured to select interested regions composed of line segments and arcs at positions of a femoral head contour line and two femoral neck contour lines of the femoral contour lines respectively.

6. An image registration method for a femur surgery, comprising the following steps of:
carrying out DRR projection at different angles of a three-dimensional femur CT image, to obtain a plurality of two-dimensional DRR projection images;
preprocessing femur images in the two-dimensional DRR projection images and two-dimensional X-ray images, to generate femoral coordinate systems and femoral characteristic points; wherein the two-dimensional X-ray image comprise a plurality of X-ray plane images taken from different angles of a femur; and
determining, from the plurality of two-dimensional DRR projection images, a two-dimensional DRR projection image in the same femoral posture as a femoral posture of the two-dimensional X-ray images according to the femoral coordinate systems and the femoral characteristic points of the two-dimensional DRR projection images and the two-dimensional X-ray images, to obtain a plurality of target DRR projection images.

7. The image registration method according to claim 6, wherein the femur comprises a femoral head, a femoral neck and a femoral shaft, and the preprocessing femur images in the two-dimensional DRR projection images and two-dimensional X-ray images, to generate femoral coordinate systems and femoral characteristic points specifically comprises:
automatically extracting a femoral outer contour, to obtain a femoral contour line;
fitting the femoral head into a circle, to obtain a femoral head fitting circle;
selecting a shortest distance connection line constituted by two points located at fitting curves of two femoral neck contour lines at the femoral neck as a femoral neck characteristic line;
establish a femoral coordinate system by using a connection line from a center of the femoral head fitting circle to a midpoint of the femoral neck characteristic line as a coordinate axis of the femoral coordinate system;
generating a first characteristic circle and a second characteristic circle respectively by using a radius of the femoral head fitting circle as a radius and selecting two points located on an extension line extended from a connection line from the center of the femoral head fitting circle to the midpoint of the femoral neck characteristic line as centers of circles, wherein the first characteristic circle is tangent to the femoral head fitting circle, and a center of the first characteristic circle is on an edge line of the second characteristic circle;
generating a plurality of femoral characteristic lines perpendicular to the connection line from the center of the femoral head fitting circle to the midpoint of the femoral neck characteristic line; and
selecting intersection points of all femoral characteristic lines and the femoral contour lines as femoral characteristic points.

8. The image registration method according to claim 7, wherein the generating a plurality of femoral characteristic lines perpendicular to the connection line from the center of the femoral head fitting circle to the midpoint of the femoral neck characteristic line specifically comprises:
selecting a line segment that passes through a tangent point of the femoral head fitting circle and the first characteristic circle as a first femoral characteristic line;
selecting a line segment that passes through the center of the first characteristic circle as a second femoral characteristic line;
selecting a line segment that passes through a center of the second characteristic circle as a third femoral characteristic line;
selecting a line segment, which is parallel to the second femoral characteristic line and is the tangent line of the second characteristic circle, as a fourth femoral characteristic line; and
selecting a line segment, which passes through a midpoint of a connection line from an intersection point of an extension line and the third femoral characteristic line to an intersection point of the extension line and the fourth femoral characteristic line, as a fifth femoral characteristic line, wherein the extension line is extended from the connection line from the center of the femoral head fitting circle to the midpoint of the femoral neck characteristic line.

9. The image registration method according to claim 6, wherein the determining, from the plurality of two-dimensional DRR projection images, a two-dimensional DRR projection image in the same femoral posture as a femoral posture of a two-dimensional X-ray image according to the femoral coordinate systems and the femoral characteristic points of the two-dimensional DRR projection images and the two-dimensional X-ray images, to obtain a plurality of target DRR projection images specifically comprises:
overlapping the femoral coordinate systems of the two-dimensional DRR projection images and the two-dimensional X-ray images; and
matching positions of the femoral characteristic points of the two-dimensional DRR projection images and the two-dimensional X-ray images in a one-to-one correspondence.

10. The image registration method according to claim 7, wherein the image registration method further comprises:
selecting interested regions composed of line segments and arcs at positions of a femoral head contour line and two femoral neck contour lines of the femoral contour lines respectively; and
identifying contour lines in the interested region, to generate fitting curves of the femoral head contour line and two femoral neck contour lines;
automatically generating a femoral head fitting circle according to the fitting curves of the femoral contour line at the femoral head; and
selecting a shortest distance connection line constituted by two points located at fitting curves of two femoral neck contour lines at the femoral nock as a femoral neck characteristic line.
